(19) United States
(12) Patent Application Publication
(10) Pub. No.: US 2013/0035601 A1
(43) Pub. Date: Feb. 7, 2013

(54) APPARATUS AND METHOD FOR GENERATING DIGITAL HEALTH SCREENING FORM

(71) Applicants: Kyu-Chang KANG, Daejeon (KR); Chang-Seok Bae, Daejeon (KR); Seong-Uk Heo, Ulsan (KR); Dong-Won Han, Daejeon (KR)

(72) Inventors: Kyu-Chang KANG, Daejeon (KR); Chang-Seok Bae, Daejeon (KR); Seong-Uk Heo, Ulsan (KR); Dong-Won Han, Daejeon (KR)

(73) Assignee: ELECTRONICS AND TELECOMMUNICATIONS RESEARCH INSTITUTE, Daegeon (KR)

(21) Appl. No.: 13/481,128

(22) Filed: May 25, 2012

(30) Foreign Application Priority Data
Aug. 3, 2011 (KR) 10-2011-0077484

(51) Int. Cl. A61B 5/0205; A61B 5/11

(52) U.S. Cl. 600/483; 600/595

(57) ABSTRACT

An apparatus for generating a digital health screening form collects sensor information including biometric information, motion information or location information of an examinee using a plurality of sensors. The apparatus determines results of preset health screening items depending on the biometric information, and then generates physical condition information including the results of the preset health screening items. The apparatus generates information about presence or absence of dyssomnia or information about physical activities, which indicates living habits of the examinee, and then generates lifelog information which includes the information about the presence or absence of dyssomnia or the information about the physical activities. The apparatus detects medical history information of the examinee in a medical history information provision server, and generates a digital health screening form containing the physical condition information, the lifelog information, and the medical history information.

### CROSS REFERENCE TO RELATED APPLICATION

This application claims the benefit of Korean Patent Application No. 10-2011-0077484, filed on Aug. 3, 2011, which is hereby incorporated by reference in its entirety into this application.

### BACKGROUND OF THE INVENTION

1. Technical Field

The present invention relates generally to an apparatus and method for generating a digital health screening form. More particularly, the present invention relates to an apparatus and method for generating a digital health screening form, which generate a digital health screening form that contains the living habits and medical history information of an examinee.

2. Description of the Related Art

A telemedicine service is a medical service for diagnosing and treating a patient located at a remote place using communication devices.

Korean Patent No. 10-0969510 and Korean Patent Application Publication No. 2010-0001610 disclose in detail medical service technologies.

However, when an examinee who receives a physical examination via such a telemedicine service is an unexpressive person or is short of medical knowledge, it may be difficult for the examinee to exactly communicate his or her symptoms to a doctor during the short hours of consultation.

Further, when receiving a physical examination from a doctor via the telemedicine service, the examinee communicates only symptoms he or she feels, that is, subjective symptoms, and thus it may be difficult for the doctor to accurately diagnose the disease or symptoms of the examinee.

### SUMMARY OF THE INVENTION

Accordingly, the present invention has been made keeping in mind the above problems occurring in the prior art, and an object of the present invention is to provide an apparatus and method for generating a digital health screening form, which provides a digital health screening form that can accurately represent the symptoms of an examinee when providing a telemedicine service to the examinee.

In accordance with an aspect of the present invention to accomplish the above object, there is provided an apparatus for generating a digital health screening form, including an information collection unit, an information analysis unit, an information conversion unit, and an information display unit. The information collection unit collects biometric information of an examinee from a body sensor, and collects motion information or location information of the examinee from a motion detection sensor. The information analysis unit determines results of preset health screening items depending on the biometric information, and then generates physical condition information including the results of the health screening items. The information conversion unit generates information about presence or absence of dyssomnia or information about physical activities, which indicates living habits of the examinee, from the biometric information, the motion information or the location information, and then generates lifelog information, which includes the information about the presence or absence of dyssomnia or the information about the physical activities. The information display unit displays a digital health screening form containing the physical condition information and the lifelog information.

In this case, the information conversion unit may generate a pattern of variation in a heart rate of the examinee in his or her sleep using heart rate information included in the biometric information, determine whether the examinee is suffering from dyssomnia depending on the heart rate variation pattern, and then generate the information about the presence or absence of dyssomnia.

Further, the information conversion unit may compare the heart rate variation pattern with a preset heart rate variation pattern, and then determine whether the examinee is suffering from dyssomnia.

In this case, the information conversion unit may generate a sleep pattern of the examinee from the motion information, determine whether the examinee is suffering from dyssomnia depending on the sleep pattern, and then generate the information about the presence or absence of dyssomnia.

In this case, the information conversion unit may calculate an amount of exercise taken by the examinee using heart rate information, included in the biometric information, and the location information and generates the information about the physical activities using the amount of exercise taken by the examinee.

Further, the information conversion unit may calculate the amount of exercise taken by the examinee by calculating movement distance or movement speed of the examinee or a heart rate of the examinee in motion from the heart rate information and the location information.

Furthermore, the information conversion unit may generate the information about the physical activities, including a number of times that the examinee exercised for a predetermined period of time, using the amount of exercise taken by the examinee.

In this case, the apparatus may further include an information link unit for searching a medical history information provision server for medical history of the examinee and medical history of a family of the examinee, and then detecting medical history information of the examinee, wherein the information display unit displays a digital health screening form further including the medical history information.

In accordance with another aspect of the present invention to accomplish the above object, there is provided a method of generating a digital health screening form, including collecting sensor information including biometric information, motion information or location information of an examinee using a plurality of sensors, determining results of preset health screening items depending on the biometric information, and then generating physical condition information including the results of the preset health screening items, generating information about presence or absence of dyssomnia or information about physical activities, which indicates living habits of the examinee, from the sensor information, and then generating lifelog information which includes the information about the presence or absence of dyssomnia or the information about the physical activities, detecting medical history information of the examinee in a medical history information provision server, and generating a digital health screening form containing the physical condition information, the lifelog information, and the medical history information.

The dyssomnia information may be information about presence or absence of dyssomnia.

In this case, the generating the lifelog information may include calculating an amount of exercise taken by the examinee by calculating movement distance or movement speed of the examinee or a heart rate of the examinee in motion from the biometric information and the location information, and generating the information about the physical activities, which includes a number of times that the examinee exercised for a predetermined period of time, using the amount of exercise taken by the examinee.

In this case, the generating the lifelog information may include generating a pattern of variation in a heart rate of the examinee in his or her sleep using the biometric information, and comparing the heart rate variation pattern with a preset heart rate variation pattern, and then generating the information about the presence or absence of dyssomnia.

In this case, the detecting the medical history information may be configured to detect the medical history information, including both anamnesis information which indicates medical history of the examinee and family history information which indicates medical history of a family of the examinee, in the medical history information provision server.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram showing the configuration of a digital health screening form system according to an embodiment of the present invention;

FIG. 2 is a diagram showing the configuration of an apparatus for generating a digital health screening form according to an embodiment of the present invention;

FIG. 3 is a diagram showing a method of displaying a digital health screening form according to an embodiment of the present invention; and

FIG. 4 is a flowchart showing a method of generating a digital health screening form according to an embodiment of the present invention.

### DESCRIPTION OF THE PREFERRED EMBODIMENTS

The present invention will be described in detail below with reference to the accompanying drawings. In the following description, redundant descriptions and detailed descriptions of known functions and elements that may unnecessarily make the gist of the present invention obscure will be omitted. Embodiments of the present invention are provided to fully describe the present invention to those having ordinary knowledge in the art to which the present invention pertains. Accordingly, in the drawings, the shapes and sizes of elements may be exaggerated for the sake of clearer description.

Hereinafter, an apparatus and method for generating a digital health screening form according to an embodiment of the present invention will be described with reference to the attached drawings.

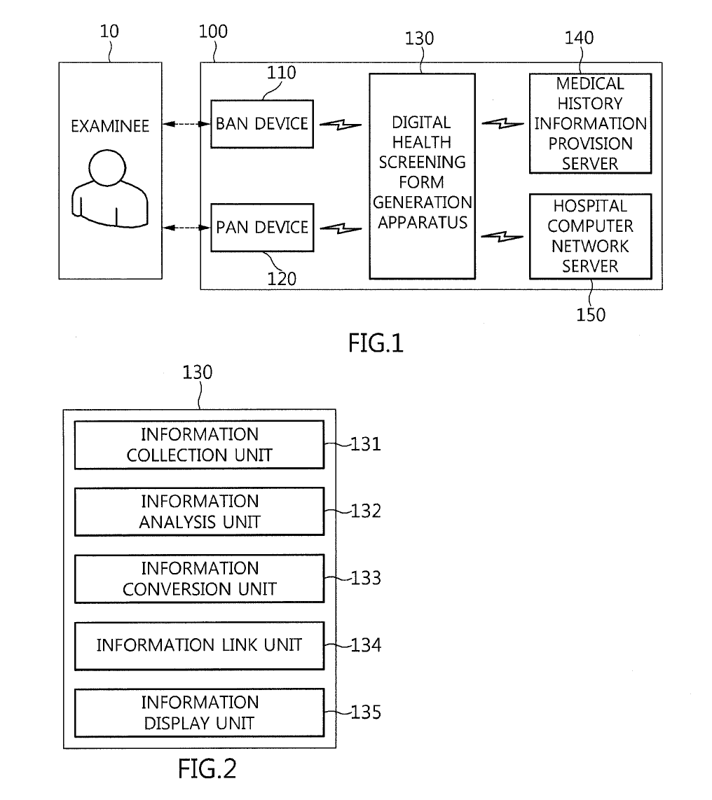

First, a digital health screening form system according to an embodiment of the present invention will be described with reference to FIG. 1.

FIG. 1 is a diagram showing the configuration of a digital health screening form system according to an embodiment of the present invention.

As shown in FIG. 1, a digital health screening form system 100 is configured to generate a digital health screening form for an examinee 10 who desires to receive a physical examination. The digital health screening form system 100 includes a body area network (hereinafter referred to as a “BAN”) device 110, a personal area network (hereinafter referred to as a “PAN”) device 120, an apparatus 130 for generating a digital health screening form (hereinafter referred to as a “digital health screening form generation apparatus”) 130, a medical history information provision server 140, and a hospital computer network server 150.

The BAN device 110 collects the biometric information of the examinee 10 while communicating with an implantable body sensor or a wearable body sensor via the BAN. The implantable body sensor is a medical sensor implanted into the body of the examinee 10 and is capable of collecting biometric information such as the blood sugar or electrocardiogram of the examinee 10. The wearable body sensor is a medical sensor attached to the outside of the body of the examinee 10 and is capable of collecting biometric information such as the body temperature, pulse, heart rate, or systolic blood pressure of the examinee 10.

In this case, the BAN device 110 not only can transmit the collected biometric information of the examinee 10 to the digital health screening form generation apparatus 130 via the PAN device 120, but also can directly transmit it to the digital health screening form generation apparatus 130. For this operation, the BAN device 110 may transmit the collected biometric information of the examinee 10 to the PAN device 120 via Bluetooth, Near-Field Communication (NFC), or the like.

The PAN device 120 is a personal portable terminal such as a mobile phone or a smart phone, has therein one or more motion detection sensors such as an acceleration sensor, a gyro sensor, or a Global Positioning System (GPS) sensor, and collects the motion information or the location information of the examinee 10 using the motion detection sensors. In this case, the PAN device 120 may collect the motion information of the examinee 10 using the acceleration sensor or the gyro sensor, and may collect the location information of the examinee 10 using the GPS sensor.

In this case, the PAN device 120 transmits the motion information or the location information of the examinee 10, which has been collected, to the digital health screening form generation apparatus 130 via a mobile communication network or a wireless Local Area Network (LAN). Here, the PAN device 120 may transmit the biometric information of the examinee 10 to the digital health screening form generation apparatus 130 when the biometric information of the examinee 10 is received from the BAN device 110.

The digital health screening form generation apparatus 130 generates information about the physical condition of the examinee 10 using the information collected by the BAN device 110, and generates a digital health screening form containing the physical condition information of the examinee 10. In this case, the digital health screening form generation apparatus 130 may determine the results of preset health screening items (questions) depending on the biometric information of the examinee 10, and may generate the physical condition information of the examinee 10 which includes the results of the preset health screening items.

The digital health screening form generation apparatus 130 generates lifelog information indicating the living habits of the examinee 10 using the information collected by the BAN device 110 and the PAN device 120, and generates a digital health screening form containing the lifelog information of the examinee 10. Here, the lifelog information may include information about the presence or absence of dyssomnia, the information about physical activities, etc.

The digital health screening form generation apparatus 130 may determine the pattern of variation in the heart rate of the examinee 10 in his or her sleep using the heart rate information of the biometric information of the examinee 10, compare the heart rate variation pattern of the examinee 10 in his or her sleep with a preset heart rate variation pattern, and then generate information about whether the examinee 10 is suffering from dyssomnia (the presence or absence of dyssomnia). Further, the digital health screening form generation apparatus 130 may generate information about whether the examinee 10 is suffering from dyssomnia, using the motion information of the examinee 10 in his or her sleep. In this case, the digital health screening form generation apparatus 130 may also generate information about whether the examinee 10 is suffering from dyssomnia, using both the heart rate information and the motion information of the examinee 10.

The digital health screening form generation apparatus 130 may calculate the amount of exercise taken by the examinee 10 based on information about the movement distance or movement speed of the examinee 10 or the heart rate of the examinee 10 in motion, using the heart rate information and the location information of the examinee 10. The apparatus 100 may generate information about physical activities, such as “the number of times that such exercise as to cause a shortness of breath lasted for 20 minutes or longer”, “the number of times that such exercise as to cause a shortness of breath lasted for 30 minutes or longer”, and “the number of days on which the examinee walked for a total of 30 minutes or longer, at 10 minutes or longer at a time”, using the calculated amount of exercise taken by the examinee 10.

The digital health screening form generation apparatus 130 generates the medical history information of the examinee 10 by searching the medical history information provision server 140 for the medical history of the examinee 10 or the medical history of the family of the examinee 10, and then generates a digital health screening form containing the medical history information of the examinee 10. Here, the medical history information of the examinee 10 may include both the anamnesis information of the examinee 10 which indicates the type or the relative seriousness of a disease or an injury previously encountered by the examinee 10 and the presence or absence of a hereditary disease or a congenital disease, and family history information which indicates the medical history of the family, near relations or cohabitants of the examinee 10.

The medical history information provision server 140 provides the medical history of the examinee 10 or the family of the examinee 10 at the request of the digital health screening form generation apparatus 130.

The hospital computer network server 150 receives the digital health screening form of the examinee 10 from the digital health screening form generation apparatus 130 and stores the digital health screening form, upon treating the examinee 10, and provides the digital health screening form of the examinee 10 to a doctor who treats the examinee 10.

In this way, although, in FIG. 1, the PAN device 120 and the digital health screening form generation apparatus 130 have been configured as separate components, the PAN device 120 and the digital health screening form generation apparatus 130 may be integrated into a single device.

Next, the digital health screening form generation apparatus according to an embodiment of the present invention will be described with reference to FIG. 2.

FIG. 2 is a block diagram showing the configuration of the digital health screening form generation apparatus according to an embodiment of the present invention.

As shown in FIG. 2, the digital health screening form generation apparatus 130 includes an information collection unit 131, an information analysis unit 132, an information conversion unit 133, an information link unit 134, and an information display unit 135.

The information collection unit 131 collects the biometric information of the examinee 10 measured by an implantable body sensor or a wearable body sensor, and collects the motion information or the location information of the examinee 10 measured by motion detection sensors.

The information analysis unit 132 analyzes the biometric information of the examinee 10 and then determines the results of preset health screening items depending on the biometric information of the examinee 10.

The information conversion unit 133 generates information about whether the examinee 10 is suffering from dyssomnia, the information about the physical activities of the examinee 10, etc. from the biometric information, motion information or location information of the examinee 10.

The information conversion unit 133 may generate the pattern of variation in the heart rate of the examinee 10 in his or her sleep from the heart rate information of the biometric information of the examinee 10, compare the generated heart rate variation pattern with a preset heart rate variation pattern, and then generate information about whether the examinee 10 is suffering from dyssomnia. Further, the information conversion unit 133 may generate the sleep pattern of the examinee 10 from the motion information of the examinee 10 in his or her sleep, and generate information about whether the examinee 10 is suffering from dyssomnia, using the generated sleep pattern. In this case, the information conversion unit 133 may also generate information about whether the examinee 10 is suffering from dyssomnia, using both the heart rate variation pattern and the sleep pattern of the examinee 10 in his or her sleep.

The information conversion unit 133 may calculate the amount of exercise taken by the examinee 10 based on the movement distance or movement speed of the examinee 10 or the heart rate of the examinee 10 in motion, using the heart rate information and the location information of the examinee 10. Further, the information conversion unit 133 may generate information about physical activities, such as “the number of times that such exercise as to cause a shortness of breath lasted for 20 minutes or longer”, “the number of times that such exercise as to cause a shortness of breath lasted for 30 minutes or longer”, and “the number of days on which the examinee walked for a total of 30 minutes or longer, at 10 minutes or longer at a time”, using the amount of exercise taken by the examinee 10.

The information link unit 134 searches the medical history information provision server 140 for the medical history of the examinee 10 or the medical history of the family of the examinee 10, and detects both anamnesis information which indicates the type or the relative seriousness of a disease or an injury previously encountered by the examinee 10 and the presence or absence of a hereditary disease or a congenital disease, and family history information which indicates the medical history of the family, near relations or cohabitants of the examinee 10.

The information display unit 135 displays the digital health screening form of the examinee 10, containing the results of the preset health screening items, information about the presence or absence of dyssomnia, physical activity information, anamnesis information and family history information, on a display device.

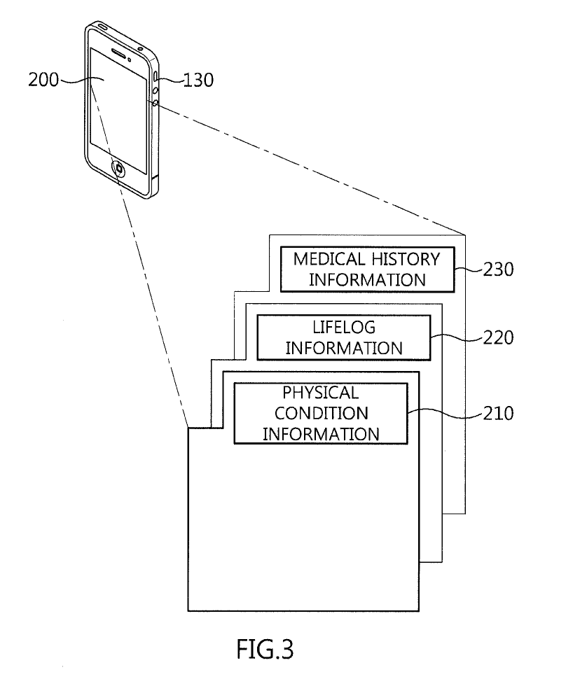

Next, a method in which the digital health screening form generation apparatus displays a digital health screening form according to an embodiment of the present invention will be described with reference to FIG. 3.

FIG. 3 is a diagram showing a method of displaying a digital health screening form according to an embodiment of the present invention.

As shown in FIG. 3, the digital health screening form generation apparatus 130 displays the digital health screening form on a display device 200.

In this case, the digital health screening form generation apparatus 130 may display the results of preset health screening items, the information about the presence or absence of dyssomnia, the physical activity information, the anamnesis information, and the family history information, which are contained in the digital health screening form of the examinee 10, on a plurality of divided screens.

Here, the digital health screening form generation apparatus 130 may display the results of the preset health screening items on the first screen 210 indicating physical condition information.

Further, the digital health screening form generation apparatus 130 may display the information about the presence or absence of dyssomnia and the physical activity information on the second screen 220 indicating lifelog information.

Furthermore, the digital health screening form generation apparatus 130 may display the anamnesis information and the family history information on the third screen 230 indicating medical history information.

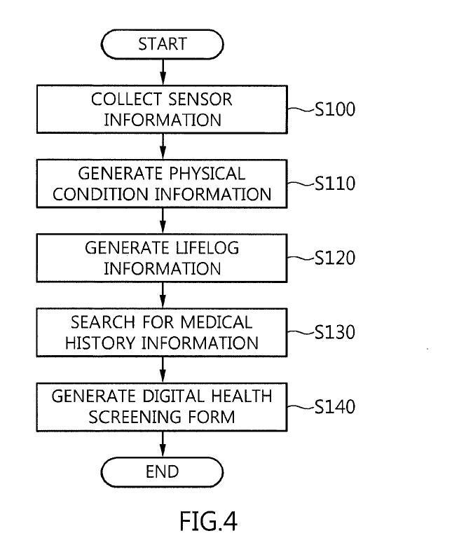

Next, a method of generating a digital health screening form according to an embodiment of the present invention will be described below with reference to FIG. 4.

FIG. 4 is a flowchart showing a method of generating a digital health screening form according to an embodiment of the present invention.

As shown in FIG. 4, the information collection unit 131 of the digital health screening form generation apparatus 130 collects the sensor information of the examinee 10 using an implantable body sensor, a wearable body sensor, and a motion detection sensor at step S100. Here, the sensor information of the examinee 10 may include the biometric information, motion information, or location information of the examinee 10.

Next, the information analysis unit 132 of the digital health screening form generation apparatus 130 determines the results of preset health screening items depending on the biometric information of the examinee 10 and then generates physical condition information including the results of the preset health screening items at step S110.

Thereafter, the information conversion unit 133 of the digital health screening form generation apparatus 130 generates lifelog information, including information about whether the examinee 10 is suffering from dyssomnia and the information about the physical activities of the examinee 10, from the biometric information, motion information or location information of the examinee 10 at step S120.

Next, the information link unit 134 of the digital health screening form generation apparatus 130 searches the medical history information provision server 140 for the medical history of the examinee 10 or the medical history of the family of the examinee 10, and then detects the medical history information of the examinee 10 at step S130. Here, the medical history information of the examinee 10 may include both the anamnesis information of the examinee 10 which indicates the type or the relative seriousness of a disease or an injury previously encountered by the examinee 10, and the presence or absence of a hereditary disease or a congenital disease, and family history information which indicates the medical history of the family, near relations or cohabitants of the examinee 10.

Thereafter, the digital health screening form generation apparatus 130 generates a digital health screening form containing the physical condition information, the lifelog information, and the medical history information of the examinee 10 at step S140.

In accordance with the features of the present invention, there is an advantage in that lifelog information incorporating the living habits of an examinee is generated, using sensor information collected by devices carried by the examinee, thus enabling a digital health screening form, into which the symptoms or habits of the examinee have been incorporated, to be generated without the intervention of the examinee when providing a telemedicine service to the examinee.

As described above, optimal embodiments of the present invention have been disclosed in the drawings and the specification. Although specific terms have been used in the present specification, these are merely intended to describe the present invention and are not intended to limit the meanings thereof or the scope of the present invention described in the accompanying claims. Therefore, those skilled in the art will appreciate that various modifications and other equivalent embodiments are possible from the embodiments. Therefore, the technical scope of the present invention should be defined by the technical spirit of the claims.

What is claimed is:

1. An apparatus for generating a digital health screening form, comprising:
an information collection unit for collecting biometric information of an examinee from a body sensor, and collecting at least one of motion information and location information of the examinee from a motion detection sensor;
an information analysis unit for determining results of preset health screening items depending on the biometric information, and then generating physical condition information including the results of the health screening items;
an information conversion unit for generating at least one of dyssomnia information and physical activity information from at least one of the biometric information, the motion information and the location information to indicate living habits of the examinee, and then generating lifelog information including at least one of the dyssomnia information and the physical activity information; and
an information display unit for displaying a digital health screening form containing the physical condition information and the lifelog information.

2. The apparatus of claim 1, wherein the information conversion unit generates a pattern of variation in a heart rate of the examinee while the examinee is sleeping using heart rate information included in the biometric information, determines whether the examinee is suffering from dyssomnia depending on the heart rate variation pattern, and then generates the dyssomnia information.

3. The apparatus of claim 2, wherein the information conversion unit compares the heart rate variation pattern with a preset heart rate variation pattern, and then determines whether the examinee is suffering from dyssomnia.

4. The apparatus of claim 1, wherein the information conversion unit generates a sleep pattern of the examinee from the motion information, determines whether the examinee is suffering from dyssomnia depending on the sleep pattern, and then generates the dyssomnia information.

5. The apparatus of claim 1, wherein the information conversion unit calculates an amount of exercise taken by the examinee using heart rate information and the location information, included in the biometric information and generates the physical activity information using the amount of exercise taken by the examinee.

6. The apparatus of claim 5, wherein the information conversion unit calculates the amount of exercise taken by the examinee by calculating at least one of movement distance, movement speed and a heart rate of the examinee in motion from the heart rate information and the location information.

7. The apparatus of claim 5, wherein the information conversion unit generates the physical activity information, including a number of times that the examinee exercised for a predetermined period of time, using the amount of exercise taken by the examinee.

8. The apparatus of claim 1, further comprising an information link unit for searching a medical history information provision server for medical history of the examinee and medical history of a family of the examinee, and then detecting medical history information of the examinee,
wherein the information display unit displays a digital health screening form further including the medical history information.

9. A method of generating a digital health screening form, comprising:
collecting sensor information including at least one of biometric information, motion information and location information of an examinee using sensors;
generating physical condition information including results of preset health screening items by determining the results of the preset health screening items depending on the biometric information;
generating lifelog information including at least one of dyssomnia information and physical activity information by generating at least one of the dyssomnia information and the physical activity information indicating living habits of the examinee, from the sensor information;
detecting medical history information of the examinee in a medical history information provision server; and
generating a digital health screening form including at least one of the physical condition information, the lifelog information and the medical history information.

10. The method of claim 9, wherein the generating the lifelog information comprises:
calculating an amount of exercise taken by the examinee by calculating at least one of movement distance, movement speed and a heart rate of the examinee in motion from the biometric information and the location information; and
generating the physical activity information including a number of times that the examinee exercised for a predetermined period of time, using the amount of exercise taken by the examinee.

11. The method of claim 9, wherein the generating the lifelog information comprises:
generating a pattern of variation in a heart rate of the examinee while the examinee is sleeping, using the biometric information; and
generating the dyssomnia information by comparing the heart rate variation pattern with a preset heart rate variation pattern.

12. The method of claim 9, wherein the detecting the medical history information is configured to detect the medical history information, including both anamnesis information indicating medical history of the examinee and family history information indicating medical history of a family of the examinee, in the medical history information provision server.
